(19) United States
(12) Patent Application Publication
(10) Pub. No.: US 2013/0079734 A1
(43) Pub. Date: Mar. 28, 2013

(54) Method and Apparatus for Applying Materials on Human Lips

(71) Applicant: Michael J. Lee, Los Angeles, CA (US)

(72) Inventor: Michael J. Lee, Los Angeles, CA (US)

(21) Appl. No.: 13/626,573

(22) Filed: Sep. 25, 2012

Related U.S. Application Data
(60) Provisional application No. 61/539,370, filed on Sep. 26, 2011.

(51) Int. Cl. A45D 40/26; A61M 35/00

(52) U.S. Cl. 604/290; 401/9; 132/200

(57) ABSTRACT

An apparatus for applying cosmetic or treatment material to a user's lips includes a container containing a lip cosmetic product or a lip treatment product in solid or semi-solid form. An applicator is mounted on the container, the applicator having an upper surface formed in the general shape of human upper and lower lips. In one embodiment, the applicator has apertures extending through the applicator. The apertures advance the product in the container through the apertures and onto the upper surface of the applicator when the container is manipulated. A user applies their lips to the upper surface of the applicator, transferring the product to the user's lips. In a further embodiment, the lip product in the container and the applicator are one solid or semi-solid structure, with the upper surface of the structure formed generally in the shape of a pair of human lips.

This application claims priority from Provisional Patent Application Ser. No. 61/539370 filed Sep. 26, 2011, to the extent allowed by law.

### FIELD OF THE INVENTION

This invention pertains generally to the application of cosmetic and medical balm products to the human lips. The present invention provides a lip shaped applicator that applies the cosmetic and balm products to the full area of the lips simultaneously and in a single “kiss” type action.

### BACKGROUND OF THE INVENTION

Human lips are very important visible parts of the mouth on human faces. Lips serve as the opening for food intake and provide varying facial expressions. Human lips comprise an upper and lower lip. The upper lip is referred in scientific terms as the “labium superius oris,” and the lower lip is referred in scientific terms as the “labium inferius oris.” A fleshy protuberance is located in the center of the upper lips and this tubercle is known by various terms including the “procheilon,” the “tuberculum labii superioris,” and the “labial tubercle.” This fleshy protuberance distinguishes the upper lip of a human from the lower lip.

In addition to the function of food intake, the lips serve in creating different sounds. Lips create mainly the labia, bilabial, and labiodental consonant sounds. Lips also enable whistling and the performance of wind and reed instruments such as the trumpet, clarinet, saxophone and the like.

The skin of the lips has three to five cellular layers, and this skin is very thin compared to typical facial skin. The lip skin does not have sweat glands or sebaceous glands, and therefore, it does not have the usual protection of sweat or body oils.

Cosmetic products have long been used to protect the skin of the upper and lower lips, and to apply color, texture and moisture to the lips. These cosmetic products include lip balms, lip salves, lip glosses, and lipsticks. These different products can be solid, semi-solid, or liquid. Lip balms may also have medicinal or soothing purposes. Lip cosmetic products can seal moisture in lips and protect the lips from dry air, cold temperatures, and offer moisturizing benefits. Lipsticks are generally worn by women to make them more attractive. The use of lipstick dates from ancient times.

These cosmetic and medicinal products are typically contained in and dispensed from a solid or squeezable tube. From the tube the product can be applied directly to the lips. These products can also be applied by using a finger to transfer the material to the lips. The materials can also be applied with a lip brush. Products that are distributed as a liquid or a semi-solid form can be filled in squeezable tubes and in wand applicator formats. The user squeezes or otherwise advances the product through an opening in the tube until the product extends from the opening, and applies the product to the lips.

A traditional way of applying cosmetic and lip balm products to the lips (vermillion zone) is to place the tube that contains the product adjacent the lips, and apply the product all the way around the reddish lip area. Still another way of applying these products to the lips is to use a brush applicator or finger to take the product from its container and apply it to the reddish lip area little by little. Still another way of applying such product is to press the product against the reddish lip area. However, there lacks a type of applicator that can simultaneously apply the product directly to the precise specific area of the lips, where the applicator fits the shape of the lips. This invention is directed to solving this problem and makes the application of lip cosmetic and medicinal product fast, easy and convenient.

It is well known to use tubes or squeezable containers to carry cosmetics or materials to be applied to lips. Lip balm can also be filled into a cylinder container and applied to the lips by the fingers or brushes. The “EOS Brand” lip balm is in a relatively solid form and has an egg shape protruding from the base of the container. The users apply this product by pressing and moving the surface of the protruding egg shaped balm over the lip surface. The Kiss-a-Zima drink topper in Japan has the shape of Japanese celebrities' lips. The topper is lipped around the neck of a bottle and makes the user feel they are kissing the celebrity when using the drink toppers.

However, there is no prior art, by itself or combed with others, that discloses an applicator having a shape analogous to human lips, or the method of using a lip shaped applicator to apply cosmetic or other material to a wide portion of the lip area.

Therefore, an object of this invention is to provide a more convenient way of applying cosmetics and other materials to the lips.

### SUMMARY OF THE INVENTION

The present invention provides an applicator for applying cosmetics or other materials onto human lips in a single action. The applicator is specifically designed to apply lip products quicker and easier to the lips.

In an embodiment, the applicator comprises a flowable, lotion or cream type lip product in a squeezable tube. An apertured applicator in the shape of a pair of human lips is mounted at the open end of the squeezable tube, with the apertures communicating with the lip product in the tube. The user squeezes the tube to advance a desired amount of the lip product through the apertures and onto the upper surface of the applicator. The user applies the lip product to substantially the entire area of the lips by contacting or kissing the applicator with the lips.

In another embodiment, the applicator is made of a solid or semisolid lip balm or cosmetic product placed in a container. The upper surface of the applicator is shaped the same as a human upper and lower lip, and a user “kisses” the lip shaped upper surface to apply the product to substantially the entire surface of the lips. In this embodiment, the lip shaped applicator retains its shape as the product in the container is sequentially used.

In an additional embodiment, the lip product is contained in a solid-walled hollow tube. The apertured lip shaped applicator is mounted at the open end of the hollow tube, which apertures communicate with the flowable lip product in the tube. A manually rotatable wheel at the bottom, closed end of the tube operates a mechanism in the tube that forces a desired amount of the lip product through the apertures and onto the surface of the lip-shaped applicator. The user then applies the lip product to substantially the entire area of the lips by contacting or kissing the applicator with the lips.

In a further embodiment, the applicator comprises a container containing a cosmetic or balm product, and an applicator having an application area that is shaped analogous to human lips. In an embodiment, the lip shaped applicator includes apertures that allow the material in the container to be conveyed from the container, through the applicator and onto the lips. The method of using this applicator is to supply the lip product through the apertures to the applicator, and the user simply kisses or presses the lips to the application area. Because the applicator has the shape of lips, products can thus be applied to substantially the entire area of the reddish lip surface simply and quickly.

In the description of the invention, the term “lip-shaped” defines shapes of the applicator that are the same as or approximately the shape of human lips. In addition, the term “lip-shaped” as used herein also defines those shapes of the applicator of the several embodiments of the invention, where the lip-shape comprises a slightly distorted or fanciful representation of a pair of human lips, but still recognizable as lips.

### BRIEF DESCRIPTION OF THE DRAWINGS

FIG. 1 is a perspective view of an embodiment of the present invention for applying materials on human lips from a squeezable tube;

FIG. 2 is a perspective view of an embodiment of the present invention for applying materials on human lips where the material to be applied to the lips is in the shape of a pair of human lips;

FIG. 3 is an exploded view of the embodiment for applying materials on human lips of FIG. 2;

FIG. 4 is a perspective view of an embodiment of the present invention for applying materials on human lips from a bottom-twist applicator tube;

FIG. 5 is a perspective schematic view of an apparatus for applying materials to human lips from a container having a lip-shaped apertured applicator;

FIGS. 6A-B are cross-section views of the apparatus for applying materials on human lips of FIG. 1, taken along lines 6A-6A and 6B-6B in FIG. 5.

### DETAILED DESCRIPTION OF THE ILLUSTRATED EMBODIMENTS

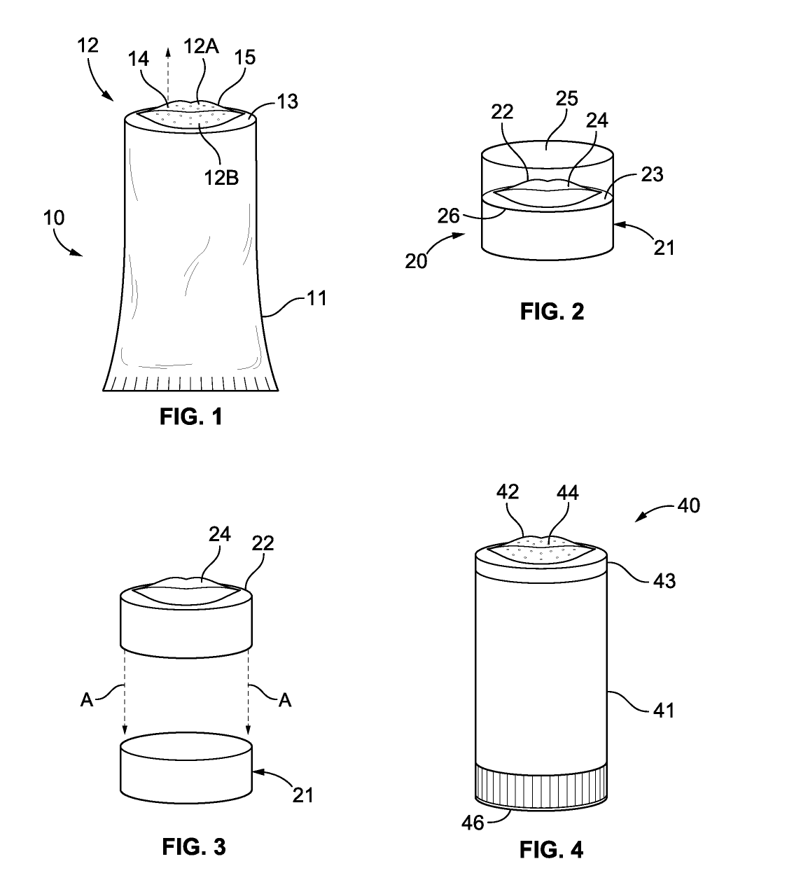

FIG. 1 is a perspective view of apparatus 10 for applying materials on human lips. In this embodiment, the lip product (not shown) is contained in squeezable tube 11. The applicator 12 is mounted on the top of the squeezable tube 10. The applicator 12 is in the form of a human lip mold 15 and has apertures 14 extending through the mold 15. The apertures 14 communicate with the lip product inside the squeezable tube 10. The applicator 12 is analogous to human lips, which have two parts, the labium superius oris 12A and the labium inferious oris 12B.

There are no specific limitations on the size or diameter of the apertures 14, so long as they can channel the lip product to reach the upper surface of lip mold 15. The applicator 12 can be mounted on the squeezable tube 11 either directly, or be mounted on a lid 13 which substantially covers the open portion at the top of squeezable tube 10. The user squeezes the tube 11 to advance the lip product in the tube through apertures 14, then applies the product by contacting or kissing the applicator 12 with the lips.

FIG. 2 is a perspective view of an apparatus 20 for applying materials on human lips in accordance with a further embodiment of the invention. In this embodiment, the applicator portion 22 is made of solid or semi-solid lip balm or cosmetic or treatment product 24 in solid or semi-solid form having an upper portion in the shape of a pair of human lips. The applicator 22 may constitute part of the container 21 or be inserted into the container. The applicator 22 comprises a solid balm block, with the lips protruding upward from the block and extending from the top of container 21. The user applies the product by contacting or kissing the applicator 22 with the lips, and the lip product 24 is transferred directly from the lip-shaped applicator 22 to the user's lips. If desired, the user may then further spread the lip product over the lips using a finger. In this embodiment, a protective lid 25 is removably connected to the upper edge 26 of container 21 by a snap-fit connection, or any other suitable connection, as are known to those skilled in the art. After many uses, the lip-shape of the balm will diminish to a flatter shape, but the balm product can still be applied to the lips.

FIG. 3 is an exploded view of the apparatus 20 shown in FIG. 2 for applying materials on human lips. The applicator 22 comprising lip product 24 is inserted into the top of the case 21 in the direction of arrows A.

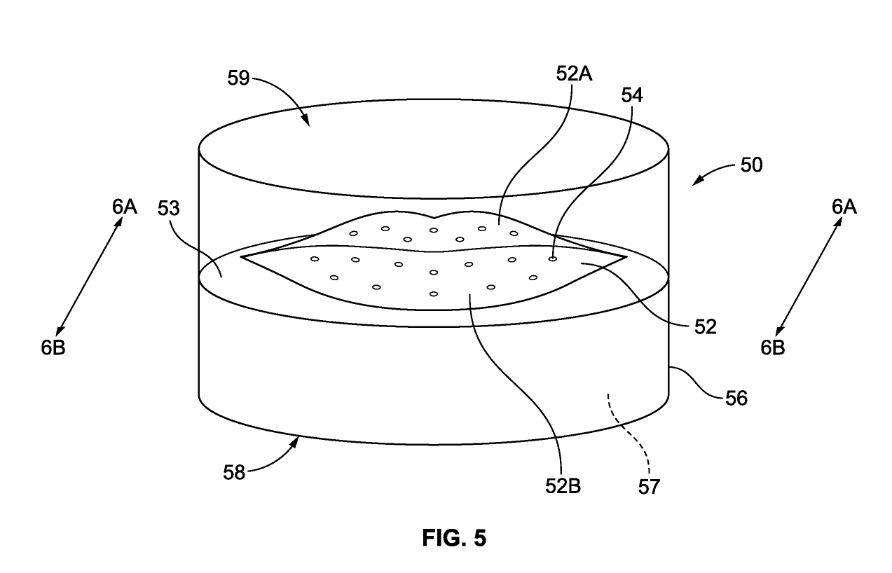

FIG. 4 is a perspective view of an apparatus 40 for applying materials on human lips in accordance with another embodiment of the invention. In this embodiment, the lip product (not shown) is contained in a hollow tube 41 having solid walls. The tube is preferably cylindrical, but may be of other shapes, if desired, such as an oval or lip shaped in cross section. The lip-shape applicator 42 is mounted on the top or open end of the hollow tube 41. The applicator 42 has apertures 44 extending through the applicator. The apertures 44 communicate with the lip product inside solid tube 41. There are no specific limitations on the size or diameter of the apertures 44 so long as the lip product can reach the upper surface of applicator 42. The applicator 42 can be mounted on the lipstick style tube either directly, or be mounted on a lid 43 which substantially covers the open upper portion of tube 41. A wheel 46 is connected to a mechanism inside tube 41 to raise the lip product and advance the lip product through apertures 44. The user manually twists wheel 46 located at the bottom of the tube 41 to force the lip product through the apertures 44 and onto the upper surface of the applicator 42. The user then applies the product by contacting or kissing the applicator 42 with the lips.

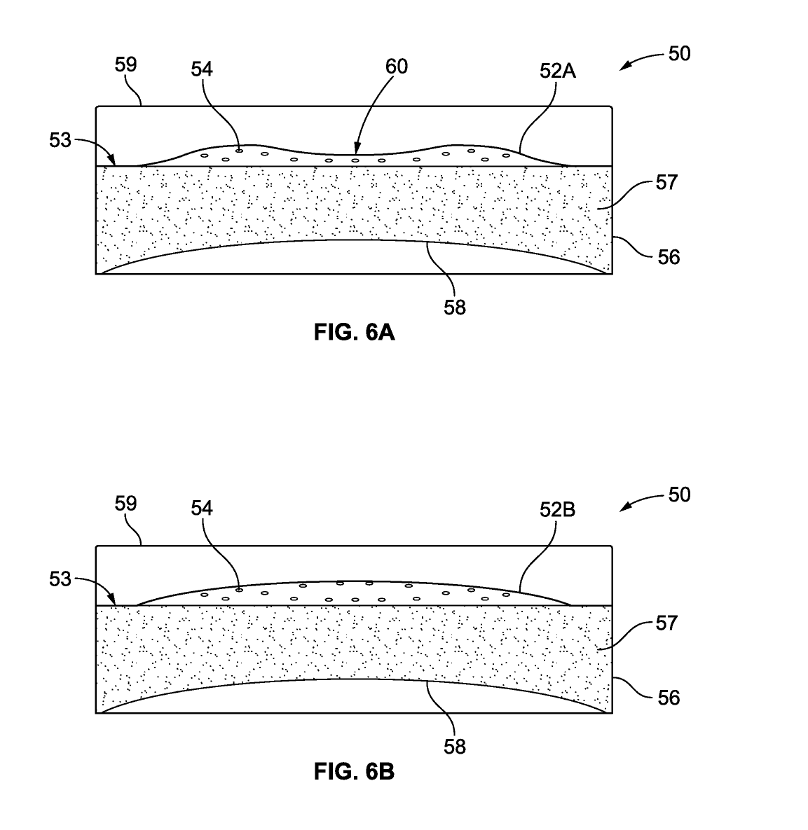

FIG. 5 is a perspective view of the apparatus 50 for applying materials on human lips in accordance with another embodiment of the invention. The apparatus 50 has an applicator 52 which is generally in the shape of the upper and lower lips of a human and made of plastic or metal, or other suitable material. The applicator 52 includes apertures 54 extending through the applicator. The applicator 52 is analogous to human lips, which have two parts, the labium superius oris 52A and the labium inferius oris 52B. The apertures 54 communicate with the cylinder-shaped container 56 which contains the lip product 57. There are no specific limitations on the size or diameter of the apertures 54, so long as they provide channels to allow the lip product 57 to migrate from the container 56 to the upper surface of applicator 52. The applicator 52 is mounted on cylindrical container 56 above the volume of lip product 57, which product can be lip gloss, lip balm, lipstick, medicinal or treatment products or the like. The applicator 52 can be mounted on the cylinder container 56 either directly, or be mounted on a lid 53 which substantially covers the cylinder container 56. The bottom 58 (FIGS. 6A-6B) of the cylinder container 56 in the illustrated embodiment comprises a flexible diaphragm-type surface that can be pushed up with a finger, so that the lip product is pushed through the apertures 54 to the upper surface of applicator 52. The user then applies the product by contacting or kissing the upper surface of the applicator 52 with the lips. A cap 59 fits over applicator 52, and makes the apparatus 50 easier to carry in a pocket or purse.

FIGS. 6A and 6B are cross-section views of the apparatus 50 for applying materials on human lips of FIG. 5. In both FIGS. 6A and 6B, the applicator portions 52A and 52B are protruding upwardly from lid 53. In an alternate embodiment (not shown), the applicator 52 can be inwardly recessed into the surface of the lid 53. FIG. 6A shows a cross-section view of the apparatus 50 viewed towards the side having the labium superious oris (upper lip) shape. The lower connecting point 60 reflects the procheilon located in the center of the labium superium oris of a human lip. FIG. 6B shows a cross section view of the apparatus 50 viewed towards the side having the labium inferius oris (the lower lip) shape. Apertures 54 connect the upper surface of the applicator 52A and 52B to the lip product 57 in the cylinder container 56. The bottom 58 of the cylinder container 56 is flexible, such that manual upward pressure on the bottom 58 forces material 57 through apertures 54 and onto the upper surface of applicators 52A and 52B. Upon applying the lips to the upper surface of the applicator, in a kissing motion, material 57 is applied to all areas of the user's lips simultaneously.

The lip mold 52A, 52B of the applicator can be made in different sizes. The shape could also be made according to the real shape of celebrities' lips. The user can pretend to “kiss” a celebrity of choice.

Specific embodiments of a method and apparatus for applying materials on human lips has been described for the purpose of illustrating the manner in which the invention is made and used. It should be understood that the implementation of other variations and modifications of the invention and its various aspects will be apparent to one skilled in the art, and that the invention is not limited by the specific embodiments described. Therefore, it is contemplated to cover the present invention and any and all modifications, variations, or equivalents that fall within the true spirit and scope of the basic underlying principles disclosed and claimed herein.

What is claimed:

1. An apparatus for applying cosmetic or treatment material on a large area of a user's lips, comprising:
a container containing one of said cosmetic and treatment material;
an applicator mounted on said container, said applicator having an upper surface formed in the shape of a labium superius oris and a labium inferius oris; and
said applicator adapted to apply said one of said cosmetic and treatment material to the lips of the user in a single motion when the user applies the user's lips to the applicator.

2. The apparatus of claim 1 wherein said applicator has at least one aperture communicating between one of said cosmetic and treatment material in said container and said upper surface of said applicator.

3. The apparatus of claim 1 wherein said applicator is made of one of solid and semi-solid material, said material being one of said cosmetic and treatment material.

4. The apparatus of claim 1 wherein said applicator is mounted on a lid, said lid substantially covering said container and said material in said container, said applicator protruding upwardly from a surface of the lid.

5. The apparatus of claim 4 wherein said applicator is recessed inwardly into the surface of said lid.

6. The apparatus of claim 1 wherein said cosmetic or treatment material is selected from the group consisting of lip gloss, lip balm, lipstick, and medicinal products.

7. The apparatus of claim 1 wherein said container is selected from the group consisting of a squeezable tube, a solid tube with means to advance the material through the tube, and a container with a flexible bottom portion.

8. A method for applying cosmetics or treatment material on a user's lips comprising:
providing the cosmetics or treatment material in a container;
mounting an applicator on the container, the applicator having an application area on an upper surface, the applicator shaped analogous to a pair of human lips;
supplying the cosmetic or treatment material to said upper surface of said applicator; and
applying the cosmetic or treatment material onto the human lips by contacting said lip shaped application area of the upper surface of the applicator with the human lips.

9. The method of claim 8 wherein the cosmetic or treatment material is supplied to the applicator by one of squeezing the container, twisting a mechanism outside the container which mechanism advances the material through the container, and applying manual pressure to the bottom of the container.

10. A method for applying cosmetic or treatment material on a user's lips comprising:
providing an applicator made of solid or semi-solid cosmetic or treatment material, said applicator having an upper surface shaped analogous to a pair of human lips;
supplying the cosmetic or treatment material to the human lips by contacting the lips to the upper surface of said applicator.

11. An apparatus for applying cosmetic or treatment material on a user's lips, comprising:
a container containing one of said cosmetic and treatment material, said container having a flexible bottom portion and an open upper portion, said material disposed against said flexible bottom portion and extending toward said open upper portion;
an applicator mounted above said open portion of said container, said applicator having an upper surface formed in the shape of a labium superius and a labium inferius oris;
said applicator having at least one aperture communicating between one of said cosmetic and treatment material and said upper surface of said applicator;
said one of said cosmetic and treatment material advancing through said at least one aperture to said upper surface of said applicator upon the application of pressure against said flexible bottom portion of said container.

12. The apparatus of claim 11 wherein:
said applicator is adapted to apply said material to the surfaces of the lips of a user when the user applies the lips to the applicator.
